(19) United States
(12) Patent Application Publication
(10) Pub. No.: US 2019/0014994 A1
(43) Pub. Date: Jan. 17, 2019

(54) MEASURING DEVICE INCLUDING LIGHT SOURCE THAT EMITS LIGHT PULSE GROUPS, PHOTODETECTOR, AND CONTROL CIRCUIT

(71) Applicant: Panasonic Intellectual Property Management Co., Ltd., Osaka (JP)

(72) Inventors: TERUHIRO SHIONO, Osaka (JP); TAKAMASA ANDO, Osaka (JP)

(21) Appl. No.: 16/013,980

(22) Filed: Jun. 21, 2018

(30) Foreign Application Priority Data
Jul. 12, 2017 (JP) 2017-136092

(51) Int. Cl. A61B 5/026; A61B 5/00; A61B 5/1455; A61B 1/00

(52) U.S. Cl. CPC A61B 5/0261; A61B 5/0075; A61B 1/00165; A61B 5/1455; A61B 5/489

(57) ABSTRACT

A measuring device includes: a light source that emits light pulse groups toward a target part of an object, the light pulse groups each including light pulses emitted sequentially; a photodetector that detects at least a part of reflected light pulse groups, the reflected light pulse groups each including reflected light pulses sequentially returning from the target part; and a control circuit that controls the light source and the photodetector. The control circuit causes the light source to emit the light pulse groups and causes the photodetector to extract a component of light included in a last reflected light pulse of the reflected light pulses included in each of the reflected light pulse groups during a falling period and output an electric signal corresponding to the component of light.

### BACKGROUND

### 1. Technical Field

The present disclosure relates to a measuring device.

### 2. Description of the Related Art

As basic parameters for determining the health condition of a human, heart rate, blood flow volume, blood pressure, oxygen saturation, and the like are widely used.

For the acquisition of biological information, electromagnetic waves falling within a wavelength range of near infrared radiation, i.e. approximately 700 nm to approximately 2500 nm, are frequently used. Among them, near infrared rays of comparatively short wavelengths, e.g. approximately not longer than 950 nm, are especially frequently used. Such near infrared rays of short wavelengths have the property of being transmitted through body tissue such as muscles, fat, and bones at comparatively high transmittances. Meanwhile, such near infrared rays have the property of being easily absorbed into oxyhemoglobin (HbO₂) and deoxyhemoglobin (Hb) in the blood. As a biological information measuring method that involves the use of these properties, near infrared spectroscopy (hereinafter abbreviated as “NIRS”) is known. Use of NIRS makes it possible to measure, for example, the amount of change in blood flow in the brain or the amounts of change in oxyhemoglobin concentration and deoxyhemoglobin concentration in the blood. It is also possible to estimate the state of activity of the brain on the basis of the amount of change in blood flow, the oxygen state of hemoglobin, or the like.

Japanese Unexamined Patent Application Publication No. 2007-260123 and Japanese Unexamined Patent Application Publication No. 2003-337102 disclose devices based on NIRS.

### SUMMARY

In one general aspect, the techniques disclosed here feature a measuring device including: a light source that emits light pulse groups toward a target part of an object, the light pulse groups each including light pulses emitted sequentially; a photodetector that detects at least a part of reflected light pulse groups, the reflected light pulse groups each including reflected light pulses sequentially returning from the target part; and a control circuit that controls the light source and the photodetector. The light pulse groups include a first light pulse group and a second light pulse group that is emitted by the light source after the first light pulse group. The reflected light pulse groups include a first reflected light pulse group and a second reflected light pulse group that returns from the target part after the first reflected light pulse group. The control circuit causes the light source to emit the light pulse groups. The control circuit causes the photodetector to extract a component of light included in a last reflected light pulse of the of reflected light pulses included in each of the reflected light pulse groups during a falling period, the falling period being a period from a point in time at which the last reflected light pulse starts decreasing in light power to a point in time at which the last reflected light pulse finishes decreasing in light power, and to output an electric signal corresponding to the component of light.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a schematic view for explaining a configuration of a biological measuring device according to Embodiment 1 of the present disclosure and the way in which a biological measurement is carried out;

FIG. 1B is a diagram schematically showing an internal configuration of a photodetector according to Embodiment 1 of the present disclosure and the flow of signals;

FIG. 2A is a diagram showing an example of a time distribution of a single light pulse that is emitted light;

FIG. 2B is a diagram showing time distributions of a total light power (solid line) of the light pulse, a power (dashed line) of light having passed through a region of change in brain blood flow, and a degree of modulation (chain line);

FIG. 3A is a diagram showing an example of a time distribution of a light pulse group that is emitted light;

FIG. 3B is a diagram showing examples of time distributions of a total light power (solid line) of the light pulse group, a power (dashed line) of light having passed through a region of change in brain blood flow, and a degree of modulation (chain line);

FIG. 4 is a diagram schematically showing examples of a time distribution (upper row) of light pulse groups, a time distribution (middle row) of a light power that is detected by the photodetector, and the timing and charge storage (lower row) of an electronic shutter;

FIG. 5A is a diagram showing examples of a relationship (dashed line) between a degree of modulation in a case where a single light pulse is used and the falling time τ of the light pulse and a relationship (solid line) between a degree of modulation in a case where a light pulse group is used and the falling time τ of the last light pulse of the light pulse group;

FIG. 5B is a diagram showing examples of a relationship (dashed line) between the degree of modulation in a case where a single light pulse is used and a frequency f and a relationship (solid line) between the degree of modulation in a case where a light pulse group is used and the frequency f;

FIG. 6A is a front view showing changes in blood flow that are present in the interior of a target part;

FIG. 6B is a cross-sectional view, taken along a Y-Z plane, showing the changes in blood flow that are present in the interior of the target part;

FIG. 7A is a diagram schematically showing changes in blood flow in the interior of the target part as obtained from an electric signal detected in the falling period of the last reflected light pulse in each reflected light pulse group;

FIG. 7B is a diagram schematically showing changes in blood flow in the interior of the target part as image-corrected by image computations;

FIG. 8A is a diagram showing an example of a time distribution of a light pulse group of P^{(a)}(f, t) where f=0.5 GHz and a=4;

FIG. 8B is a diagram showing an example of a time distribution of a light pulse group of P^{(a)}(f, t) where f=0.5 GHz and a=0.2;

FIG. 9 is a diagram showing an example of a relationship between the full width at half maximum and degree of modulation of each light pulse of a light pulse group of P^{(a)}(f, t) where f=0.25 GHz;

FIG. 10A is a schematic view for explaining a configuration of a biological measuring device according to Embodiment 2 and the way in which a biological measurement is carried out;

FIG. 10B is a diagram schematically showing an internal configuration of a photodetector according to Embodiment 2 and the flow of signals;

FIG. 11 is a diagram schematically showing a time distribution (upper row) of light pulse groups, a time distribution (middle row) of a light power that is detected by the photodetector, and the timing and charge storage (lower row) of an electronic shutter according to Embodiment 2;

FIG. 12 is a diagram schematically showing a time distribution (upper row) of light pulse groups, a time distribution (middle row) of a light power that is detected by the photodetector, and the timing and charge storage (lower row) of the electronic shutter according to a modification of Embodiment 2;

FIG. 13A is a diagram showing examples of a time distribution (upper row) of LD driving voltage and a time distribution (lower row) of detected voltage of a photodetector at the time of emission of a plurality of light pulse groups;

FIG. 13B is a diagram showing an example of a time distribution of one of the plurality of light pulse groups shown in FIG. 13A;

FIG. 13C is a diagram showing examples of a time distribution (upper row) of LD driving voltage and a time distribution (lower row) of detected voltage of a photodetector in a case where a single light pulse is emitted; and

FIG. 13D is an enlarged view of temporal changes in detected voltage in the lower row of FIG. 13B and temporal changes in detected voltage in the lower row of FIG. 13C.

### DETAILED DESCRIPTION

Prior to a description of embodiments of the present disclosure, underlying knowledge forming the basis of the present disclosure is described.

Japanese Unexamined Patent Application Publication No. 2007-260123 discloses an endoscopic device based on NIRS. The endoscopic device disclosed in Japanese Unexamined Patent Application Publication No. 2007-260123 uses light pulses as illuminating light to observe blood flow information in blood vessels buried in body tissue covered with visceral fat. In so doing, by making an imaging timing later than the timing of incidence of a light pulse, imaging of intense noise light that returns temporally early is avoided. This improves the signal-to-noise ratio (S/N ratio) of signal light returning from a deep place in the body tissue.

Japanese Unexamined Patent Application Publication No. 2003-337102 discloses a biological activity measuring device based on NIRS. This measuring device includes a light source section that generates infrared light, a photodetection section that detects infrared light from a target part of a living body, and a controller. This measuring device measures brain functions in a non-contact manner.

The device disclosed in Japanese Unexamined Patent Application Publication No. 2003-337102 makes it possible to measure brain activity by means of NIRS. However, since light reflected by the target part includes intense noise light that returns temporally early, the S/N ratio of a signal that is detected is undesirably low.

It is conceivable that this problem may be solved by combining the technology of Japanese Unexamined Patent Application Publication No. 2007-260123 with the device of Japanese Unexamined Patent Application Publication No. 2003-337102. That is, it is conceivable that the influence of intense noise light that returns temporally early can be curbed by making the timing of detection of light later than the timing of incidence of a light pulse.

However, the inventors studied and found that, even with such measures being taken, it is difficult to make the S/N ratio sufficiently high. Emitted light having entered the brain scatteringly propagates through the brain. Detection of the light makes it possible to acquire information on blood flow in the brain. However, on the optical path from inside the brain to the device, i.e. on a return path, the light always passes through a region of distribution of blood flow near the surface of the living body, i.e. scalp blood flow. Therefore, information on scalp blood flow, as well as the information on brain blood flow, is greatly superimposed onto the light. As a result of that, the S/N ratio of a detection signal of the returning light deteriorates, so that accurate information on brain blood flow is hardly obtained. That is, with a method based on the combination of the conventional technologies, it is impossible to make the S/N ratio of the detection signal sufficiently high.

The inventors have found the foregoing problems and conceived of a novel measuring device.

The present disclosure encompasses measuring devices according to the following items.

### Item 1

A measuring device according to Item 1 of the present disclosure includes: a light source that emits light pulse groups toward a target part of an object, the light pulse groups each including light pulses emitted sequentially;

a photodetector that detects at least a part of reflected light pulse groups, the reflected light pulse groups each including reflected light pulses sequentially returning from the target part; and

a control circuit that controls the light source and the photodetector.

The light pulse groups include a first light pulse group and a second light pulse group that is emitted by the light source after the first light pulse group. The reflected light pulse groups include a first reflected light pulse group and a second reflected light pulse group that returns from the target part after the first reflected light pulse group. The control circuit causes the light source to emit the light pulse groups, and

the control circuit causes the photodetector to extract a component of light included in a last reflected light pulse of the reflected light pulses included in each of the reflected light pulse groups during a falling period, the falling period being a period from a point in time at which the last reflected light pulse starts decreasing in light power to a point in time at which the last reflected light pulse finishes decreasing in light power, and to output an electric signal corresponding to the component of light.

An example of the object is a living body, food, or the like.

### Item 2

In the measuring device according to Item 1, the control circuit may first cause the light source to emit the first light pulse group and then, after a period of time has elapsed, cause the light source to emit the second light pulse group, the period of time being longer than a period of time from beginning to end of emission of two consecutive light pulses included in the light pulse groups.

### Item 3

In the measuring device according to Item 1 or 2, the control circuit may cause the light source to emit the light pulses in each of the light pulse groups at a frequency f, and

the frequency f is equal to or higher than 0.25 GHz.

### Item 4

In the measuring device according to Item 3, 0.5/f≤pw<1/f may be satisfied where pw is a full width at half maximum of a light power of each of the light pulses included in each of the light pulse groups.

### Item 5

In the measuring device according to any one of Items 1 to 4, the photodetector may be an image sensor including photodetection cells arrayed two-dimensionally,

each of the photodetection cells may accumulate the component of light as a signal charge, and

the electric signal may correspond to the signal charge accumulated by each of the photodetection cells.

### Item 6

In the measuring device according to any one of Items 1 to 5, the first light pulse group may include light pulses having a wavelength of not shorter than 650 nm to shorter than 805 nm,

the second light pulse group may include light pulses having a wavelength of longer than 805 nm to not longer than 950 nm, and

the control circuit may cause the light source to alternately emit the first light pulse group and the second light pulse group.

### Item 7

In the measuring device according to any one of Items 1 to 5, the first light pulse group may include light pulses having a wavelength of not shorter than 650 nm to shorter than 805 nm,

the second light pulse group may include light pulses having a wavelength of longer than 805 nm to not longer than 950 nm, and

the control circuit may cause the light source to emit either one of the first and second light pulse groups at least once within a first period and cause the light source to emit the other one of the first and second light pulse groups at least once within a second period that follows the first period.

### Item 8

In the measuring device according to any one of Items 1 to 7, the light source may be a semiconductor laser, and

by supplying the light source with a driving current on which a high-frequency component has been superimposed, the control circuit may cause the light source to emit the light pulse groups.

### Item 9

In the measuring device according to any one of Items 1 to 7, the light source may be a self-oscillation laser. In the measuring device according to any one of Items 1 to 9, the detector may include an electronic shutter, and the control circuit may close the electronic shutter until the falling period starts and open the electronic shutter after the falling period starts.

In the present disclosure, all or some of the circuits, units, devices, members, or sections or all or some of the functional blocks in the block diagrams may be implemented as one or more of electronic circuits including, but not limited to, a semiconductor device, a semiconductor integrated circuit (IC), or an LSI (large scale integration). The LSI or IC can be integrated into one chip, or also can be a combination of multiple chips. For example, functional blocks other than a memory may be integrated into one chip. The name used here is LSI or IC, but it may also be called system LSI, VLSI (very large scale integration), or ULSI (ultra large scale integration) depending on the degree of integration. A Field Programmable Gate Array (FPGA) that can be programmed after manufacturing an LSI or a reconfigurable logic device that allows reconfiguration of the connection or setup of circuit cells inside the LSI can be used for the same purpose.

Further, it is also possible that all or some of the functions or operations of the circuits, units, devices, members, or sections are implemented by executing software. In such a case, the software is recorded on one or more non-transitory recording media such as a ROM, an optical disk, or a hard disk drive, and when the software is executed by a processor, the software causes the processor together with peripheral devices to execute the functions specified in the software. A system or device may include such one or more non-transitory recording media on which the software is recorded and a processor together with necessary hardware devices such as an interface.

In the following, embodiments of the present disclosure are more specifically described. Note, however, that an unnecessarily detailed description may be omitted. For example, a detailed description of a matter that has already been well known and a repeated description of substantially identical configurations may be omitted. This is intended to prevent the following description from becoming unnecessarily redundant and facilitate understanding of persons skilled in the art. It should be noted that the inventors provide the accompanying drawings and the following description so that persons skilled in the art can sufficiently understand the present disclosure, and do not intend to thereby limit the subject matters recited in the claims. In the following description, identical or similar constituent elements are given the same reference signs.

In the following, embodiments are described with reference to the drawings.

### EMBODIMENT 1

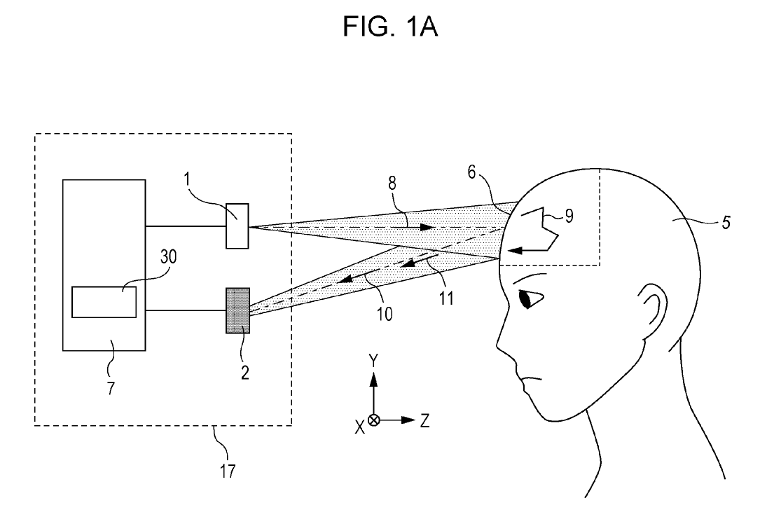

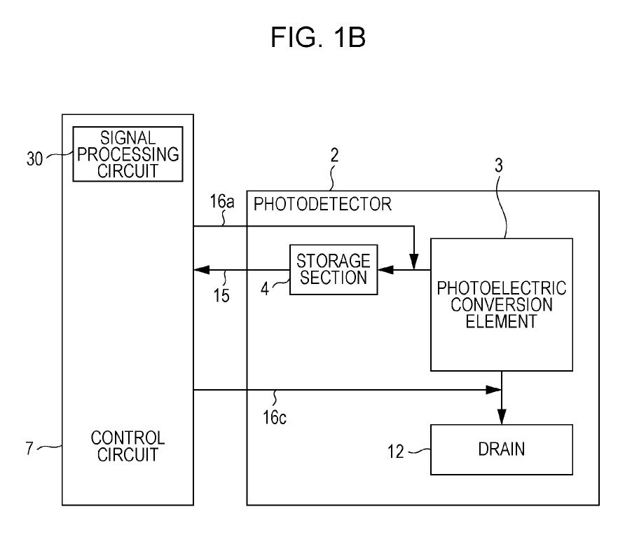

FIG. 1A is a schematic view for explaining a configuration of a biological measuring device 17 according to Embodiment 1 of the present disclosure and the way in which a biological measurement is carried out. FIG. 1B is a diagram schematically showing an internal configuration of a photodetector 2 according to Embodiment 1 of the present disclosure and the flow of signals.

The biological measuring device 17 according to Embodiment 1 includes a light source 1, the photodetector 2, and a control circuit 7 that controls the light source 1 and the photodetector 2.

The light source 1 and the photodetector 2 are arranged side by side. The light source 1 emits light toward a target part 6 of a subject 5. The photodetector 2 detects light emitted from the light source 1 and reflected by the target part 6. The control circuit 7 controls the emission of light by the light source 1 and the detection of light by the photodetector 2. The biological measuring device 17 according to Embodiment 1 includes a signal processing circuit 30 that processes electric signals (hereinafter simply referred to as “signals”) that are outputted from the photodetector 2. The signal processing circuit 30 performs computations based on a plurality of signals outputted from the photodetector 2 and thereby generates information about blood flow in the interior of the target part 6.

The target part 6 according to Embodiment 1 is a forehead part of the subject 5. Information on brain blood flow can be acquired by irradiating the forehead part with light and detecting the resulting scattered light. The “scattered light” includes reflected scattered light and transmitted scattered light. In the following description, the reflected scattered light is sometimes simply referred to as “reflected light”.

Present in the interior of the forehead, which is the target part 6, are the scalp (approximately 3 to 6 mm thick), the skull (approximately 5 to 10 mm thick), the cerebrospinal fluid layer (approximately 2 mm thick), and the brain tissue, starting from the surface. The ranges of thicknesses in parentheses mean that there are differences between individuals. Blood vessels are present in the scalp and in the brain tissue. Therefore, blood flow in the scalp is called “scalp blood flow”, and blood flow in the brain tissue is called “brain blood flow”. In a brain function measurement according to Embodiment 1, a measurement object is a target part where there is a blood flow distribution in the interior of the scalp.

A living body is a scatterer. A portion of light 8 emitted toward the target part 6 returns as directly-reflected light 10 to the biological measuring device 17. Another portion of the light enters the interior of the target part 6 and gets diffused, and a portion of it is absorbed. A portion of the light having entered the interior of the target part 6 turns into internally-scattered light 9 including information on blood flow that is present in a range of depth of approximately 10 to 18 mm from the surface, i.e. brain blood flow. The internally-reflected light 9 returns to the biological measuring device 17 as reflected scattered light 11 from the interior. The directly-reflected light 10 and the reflected scattered light 11 from the interior are detected by the photodetector 2.

It takes a relatively shorter time and a relatively longer time for the directly-reflected light 10 and the reflected scattered light 11 from the interior, respectively, to arrive at the photodetector 2 after being emitted from the light source 1. Among them, the component required to be detected at a high S/N ratio is the reflected scattered light 11 from the interior, which has the information on brain blood flow.

It should be noted that the transmitted scattered light, as well as the reflected scattered light, may be used in carrying out a biological measurement other than a brain blood flow measurement. In a case where information on blood other than brain blood flow is acquired, the target part 6 may be a part other than the forehead (e.g. an arm, a leg, or the like). In the following description, unless otherwise noted, the target part 6 is the forehead. The subject 5 is a human, but may alternatively be a non-human animal having skin and having a hairless part. The term “subject” as used herein means specimens in general including such animals.

The light source 1 emits light of, for example, not shorter than 650 nm to not longer than 950 nm. This wavelength range is included in a wavelength range of red to near infrared radiation. The aforementioned wavelength range is called “biological window” and known to be low in absorptance in the body. The light source 1 according to Embodiment 1 is described as one that emits light falling within the aforementioned wavelength range, but light falling within another wavelength range may be used. The term “light” as used herein means not only visible light but also infrared radiation.

In a visible light region of shorter than 650 nm, absorption by hemoglobin in the blood is high, and in a wavelength range of longer than 950 nm, absorbance by water is high. Meanwhile, in a wavelength range of not shorter than 650 nm to not longer than 950 nm, the absorption coefficients of hemoglobin and water are comparatively low and the scattering coefficients of hemoglobin and water are comparatively high. Therefore, light falling within the wavelength range of not shorter than 650 nm to not longer than 950 nm is subjected to strong scattering after entering the body and returns to the body surface. This makes it possible to efficiently acquire information on the interior of the body. Accordingly, Embodiment 1 mainly uses light falling within the wavelength range of not shorter than 650 nm to not longer than 950 nm.

The light source 1 may be a laser light source, such as a laser diode (LD), that repeatedly emits a light pulse. In a case where the subject 5 is a human as in the case of Embodiment 1, the impact of the light 8 on the retina is considered. In a case where a laser light source is used as the light source 1, a laser light source that satisfies Class 1 of laser safety standards devised by each country is selected, for example. In a case where Class 1 is satisfied, light of such low illuminance that the accessible emission limit AEL falls below 1 mW is emitted toward the part being test 6 of the subject 5. Since the light is of low illuminance, the sensitivity of the photodetector 2 is not enough in many cases. In that case, a light pulse is repeatedly emitted. It should be noted that the light source 1 per se does not need to satisfy Class 1. For example, light is diffused or attenuated by placing an element such as a diffusing plate or an ND filter between the light source 1 and the target part 6. In this way, Class 1 of the laser safety standards may be satisfied.

An optical element such as a lens may be provided on an emission surface of the light source 1 to adjust the degree of divergence of the light 8. Furthermore, an optical element such as a lens may be provided on a light-receiving surface side of the photodetector 2 to adjust the rate of extraction of reflected scattered light that is received.

The light source 1 is not limited to a laser light source but may be another type of light source such as a light-emitting diode (LED). Widely useable examples of the light source 1 include a semiconductor laser, a solid laser, a fiber laser, a super luminescent diode, an LED, and the like.

The light source 1 can start and stop the emission of a light pulse and change light powers in accordance with instructions from the control circuit 7. This allows almost any light pulse to be generated from the light source 1.

The photodetector 2 detects light returning from the target part 6. The photodetector 2 may include a single photodetection element or may include a plurality of photodetection elements arrayed one-dimensionally or two-dimensionally. FIG. 1B schematically shows a configuration of one photodetection element in the photodetector 2. The photodetection element of the photodetector 2 in this example includes a photoelectric conversion element 3 that generates signal charge corresponding to the amount of light received, a storage section 4 in which signal charge is accumulated, and a drain 12 through which signal charge is discharged. The photoelectric conversion element 3 may include, for example, a photodiode. Signal charge produced by the photoelectric conversion element 3 is accumulated in the storage section 4 or discharged through the drain 12. The timings of signal storage and discharge are controlled by the control circuit 7 and an internal circuit of the photodetector 2. The internal circuit of the photodetector 2 involved in this control is herein sometimes referred to as “electronic shutter”.

The photodetector 2 may be an image sensor having sensitivity to light in a wavelength range including wavelengths of light that is emitted from the light source 1. An example of such an image sensor may be a CCD or CMOS image sensor. Use of an image sensor makes it possible to acquire information on a two-dimensional intensity distribution of light. In a case where the photodetector 2 is an image sensor, the photodetector 2 includes a plurality of photodetection cells arrayed two-dimensionally. As shown, for example, in FIG. 1B, each of the photodetection cells includes constituent elements such as the photoelectric conversion element 3 and the storage section 4.

As will be mentioned later, use of the photodetector 2 such as an image sensor makes it possible to generate an image that indicates a state of brain blood flow. One image may be generated by repeating light emission and signal charge storage more than once within one frame period. A moving image can be generated by repeatedly executing such image generation every predetermined frame period.

In order to quantify the light amounts of the directly-reflected light 10 and the reflected scattered light 11, the inventors ran a simulation of a light pulse response assuming, as the target part 6, a phantom mimicking the head of a typical Japanese. Specifically, the inventors calculated through a Monte Carlo analysis a time distribution of a light power, i.e. a light pulse response, that is detected by the photodetector 2 in a case where a light pulse is emitted toward the target part 6 located at a distance of, for example, 15 cm from the light source 1.

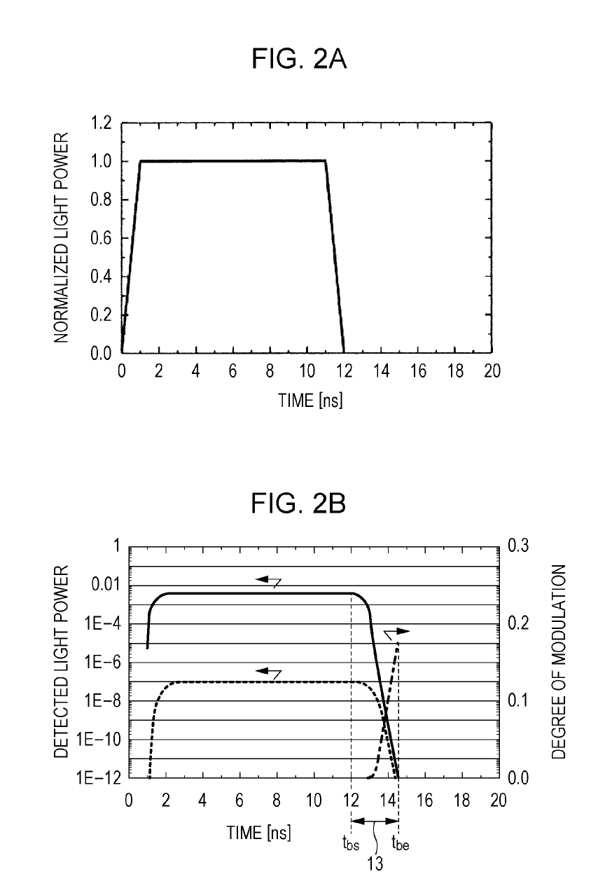

FIG. 2A is a diagram showing an example of a time distribution of a single light pulse that is emitted light. In this example, the light pulse has a wavelength 80 of 850 nm and a full width at half maximum of 11 ns. This light pulse has a typical trapezoidal shape whose rising and falling times are each 1 ns. The term “rising time” as used herein means the time it takes for a light power to increase from 0% to 100% of its peak value, and the period is referred to as “rising period”. The term “falling time” as used herein means the time it takes for a light power to decrease from its peak value to zero, and the period is referred to as “falling period”. The example shown in FIG. 2A assumes that the emission of the single light pulse starts at a time t=0 and completely stops at t=12 ns.

Since the velocity of light c is 300000 km/s and the distance from the light source 1 to the target part 6 is 15 cm, the time t from the emission of the light 8 to the arrival of the light 8 at the surface of the target part 6 is 0.5 ns. The time it takes for the light 8 to arrive at a surface of the photodetector 2 after being directly reflected by the surface of the target part 6 and turning into the directly-reflected light 10 is expressed as t=1 ns. Therefore, the period during which the photodetector 2 detects light is expressed as t=1 ns or later.

The biological measuring device 17 measures the amount of change in light amount of the reflected scattered light 11 from the interior of the target part 6 on the basis of changes in oxyhemoglobin concentration and deoxyhemoglobin concentration in the brain blood flow. The brain tissue has an absorber whose absorption coefficient and scattering coefficient vary according to changes in brain blood flow. In a stationary state, it is possible to model the interior of the brain as uniform brain tissue and conduct a Monte Carlo analysis. The term “changes in blood flow” as used herein means temporal changes in blood flow. The term “stationary state” here means a state where the subject is comparatively stable in brain activity and there are no sudden time fluctuations in brain activity of the subject.

FIG. 2B is a diagram showing time distributions of a total light power (solid line) of the light pulse, a power (dashed line) of light having passed through a region of change in brain blood flow, and a degree of modulation (chain line). The degree of modulation means a value obtained by dividing, by the total amount of light, the amount of light having passed through the region of change in brain blood flow. In FIG. 2B, the vertical axis is expressed by a logarithmic display. The amount of light having passed through the region of change in brain blood flow, which is included in the total amount of light that is detected by the photodetector 2, is only approximately 2×10⁻⁵.

Let it be assumed that t_{bs} is the time at which the light power starts to decrease on the photodetector 2 and t_{be} is the time at which the light power completely decreases to a noise level. In the example shown in FIG. 2B, the falling time is expressed as t_{f}=t_{be}−t_{bs}. t_{f} t is longer than the falling time τ of a light pulse that is emitted from the light source 1 (t_{f}>τ). As shown in FIG. 2B, it is found that the proportion of signals that indicate changes in brain blood flow becomes higher in a falling period 13 of light (t_{bs}≤t≤t_{be}). As the second half of the falling period 13 passes, the light amount decreases and noise increases accordingly. However, the degree of modulation becomes higher. Of the light falling period 13, the amount of light at and after t=13.5 ns, for example, is approximately 1/100 of the total amount of light detected. In a case where light arriving during the falling period 13 is detected with use of the function of an electronic shutter of the photodetector 2, the proportion of the light having passed through the region of change in brain blood flow increases to 7% of the total amount of light detected at and after t=13.5 ns. This makes it possible to sufficiently acquire signals that indicate changes in brain blood flow. Without use of the electronic shutter, the proportion of changes in brain blood flow is approximately 2×10⁻⁵.

Therefore, signals that indicate changes in brain blood flow can be detected by using the photodetector 2 to receive a component of the reflected scattered light 11 included in the falling period 13 of light from the target part 6 and detect changes in light amount thereof.

The inventors conceived of, acquiring blood flow information by, instead of using a single light pulse such as that shown in FIGS. 2A and 2B, using a light pulse group including a plurality of light pulses that follow one after the other. By detecting a rear-end part of a light pulse group with a photodetector, temporal changes in brain blood flow of the target part can be detected with a higher S/N ratio than in a case where a single light pulse is used. Furthermore, the quality of a signal corresponding to a state of brain blood flow can be improved by detecting a front-end part of a light pulse group and correcting a signal of the rear-end part with use of a signal of the front-end part. As will be mentioned later, use of a light pulse group also brings about an effect of making it possible to reduce speckle noise contained in a signal.

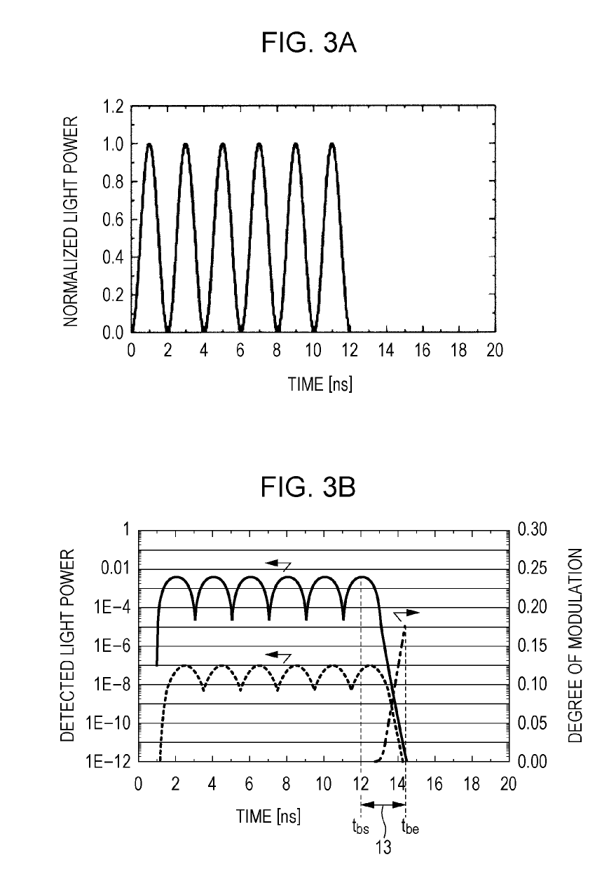

FIG. 3A is a diagram showing an example of a time distribution of a light pulse group that is emitted light. In the example shown in FIG. 3A, the light pulse group includes six light pulses each having a full width at half maximum of 1 nanosecond. The number of light pulses that are included in one light pulse group does not need to be 6 and is not limited to a particular number. The power of a plurality of light pulses in a light pulse group according to Embodiment 1 periodically varies at a frequency f. In the example shown in FIG. 3A, since the rising time and falling time of each pulse is each expressed as τ=1 ns and the cycle period of light pulses is expressed as Λ=2τ=2 ns, the frequency is expressed as f=1/Λ=0.5/τ=0.5 GHz. Without being limited to this example, the frequency of light pulses may be set at any value. The frequency of light pulses may for example be expressed as f≥0.25 GHz. The normalized light power of the light pulse group in this exampled is expressed as P(f, t)=0.5{1−cos(2πft)}. In the example shown in FIG. 3, the emission of the light pulse group starts at the time t=0 and completely stops at the time t=12 ns.

In a case where the light source 1 is a semiconductor laser light source, the control circuit 7, by supplying the light source 1 with a driving current on which a high-frequency component has been superimposed, can cause the light source 1 to emit one or more light pulse groups. In this case, the control circuit 7 may include a separate driving circuit for supplying a high-frequency current. The light source 1 may be a self-oscillation laser light source. In that case, the control circuit 7 may supply the light source 1 with a DC driving current.

FIG. 3B is a diagram showing examples of time distributions of a total light power (solid line) of the light pulse group, a power (dashed line) of light having passed through a region of change in brain blood flow, and a degree of modulation (chain line). As will be mentioned later, the inventors found that a higher degree of modulation can be attained in the example shown in FIG. 3B than in the example shown in FIG. 2B. Furthermore, a light pulse group has the following advantage over a single light pulse. Irradiation of the target part 6 with a single light pulse leads to the appearance of a pattern of bright and dark spots in the area being irradiated. This is called speckle noise, which causes deterioration in measurement accuracy. On the other hand, irradiation of the target part 6 with a light pulse group leads to a reduction in such speckle noise.

The following describes an example of a biological measuring method based on the aforementioned principle of measurement of changes in brain blood flow.

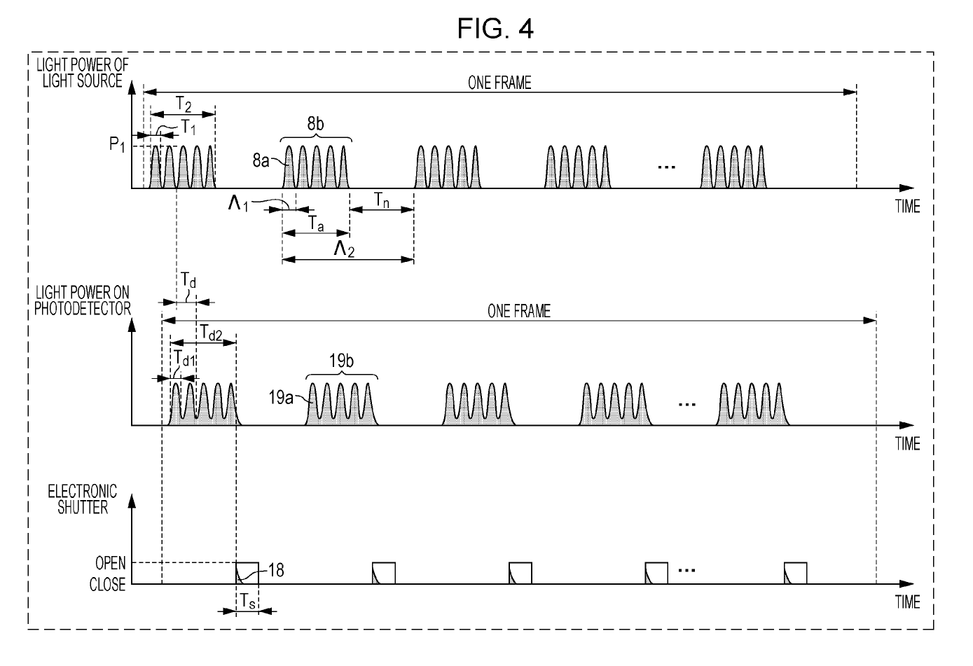

FIG. 4 is a diagram schematically showing examples of a time distribution (upper row) of light pulse groups 8b, a time distribution (middle row) of a light power that is detected by the photodetector 2, and the timing and charge storage (lower row) of an electronic shutter.

In this example, the control circuit 7 causes the light source 1 to emit a plurality of light pulse groups 8b within one frame period. The control circuit 7 causes the photodetector 2 to detect, within one frame period, a component of light included in the falling period 13 of the last light pulse in each light pulse group 8b reflected by the target part 6 and output an electric signal corresponding to the component of light. The electric signal represents a total amount of the component of light accumulated within one frame period.

As shown in the upper row of FIG. 4, the light source 1 emits a light pulse group 8b more than once within one frame period. There is a pause period Tₙ between two consecutive light pulse groups 8b. The leading light pulse 8a in each of the light pulse groups 8b has a pulse width T₁ and a maximum light power value P₁. Each of the light pulse groups 8b has a pulse width T₂. The term “pulse width” as used herein means the full width at half maximum of a pulse waveform. Each of the light pulse groups 8b in this example includes five light pulses.

As shown in the middle row of FIG. 4, reflected light pulses 19a returning from the target part 6 due to the leading light pulses 8a each have a pulse width T_{d1}, which is substantially the same as T₁. Similarly, reflected light pulse groups 19b returning from the target part 6 due to the light pulse groups 8b each have a pulse width T_{d2}, which is substantially the same as T₂. As shown in the middle row of FIG. 4, the reflected light pulses 19a have shapes that become slightly wider toward the skirts due to the occurrence of time delays under the influence of internal scattering.

The photodetector 2 uses the photoelectric conversion element 3 thereof to photoelectrically convert the component of light included in the falling period 13 of the last reflected light pulse in a reflected light pulse group 19b and uses the storage section 4 to accumulate a signal charge 18.

In a case where the target part 6 is the forehead of a human, a light pulse group 8b may enter the eyes. For this reason, a light pulse group 8b may be emitted, for example, with such a low power as to satisfy Class 1. In order to secure such a sufficient amount of light that the photodetector 2 can detect, a light pulse group 8b may be repeatedly emitted. Let it be assumed that Λₗ is the time from the beginning to end of emission of a leading light pulse 8a, Tₐ is the time from the beginning to end of emission of a light pulse group 8b, and Tₙ is the time from the end of emission of a light pulse group 8b to the beginning of emission of the next light pulse group 8b. In the example shown in FIG. 4, Tₐ=5Λ₁. In an example, a light pulse group 8b is repeatedly emitted approximately 10000 times to 1000000 times in a time cycle Λ₂=Tₐ+Tₙ of approximately 55 ns to 110 ns.

The control circuit 7 according to Embodiment 1 first causes the light source 1 to emit one light pulse group and then, after a period of time that is longer than a period of time (i.e. 2Λ₁) from the beginning to end of the emission of two consecutive light pulses included in the plurality of light pulse groups has elapsed, causes the light source 1 to emit the next light pulse group. That is, a pause period that is longer than the duration of two light pulses exists between two consecutive light pulse groups. In the example shown in FIG. 4, the control circuit 7 causes the light source 1 to emit each light pulse group by emitting a plurality of light pulses at the frequency f and causes the light source 1 to emit a plurality of light pulse groups at a frequency of f/4 or lower. A light pulse group 8b may include more or less than five light pulses. For example, each light pulse group 8b may include two light pulses. In that case, Tₐ=2Λ₁, and Λ₂ may be set to be four or more times longer than Λ₁. By thus securing a sufficient pause period between two consecutive light pulse groups, an overlap between the falling period of the last light pulse in one light pulse group and the rising period of the first light pulse in the next light pulse group can be avoided in the time distribution of the light power on the photodetector 2 as shown in the middle row of FIG. 4.

The photodetector 2 in this example is an image sensor that can measure a two-dimensional distribution of light power. The photodetector 2 generates a frame every certain frame period. One frame is composed by the charge accumulated in the storage section 4 of each of the photodetection elements of the photodetector 2. A frame is image data that represents a state of brain blood flow in the target part 6. By arranging a plurality of frames on a time-series basis, a moving image that indicates temporal changes in state of brain blood flow can be composed.

It is possible, without imposing a Class 1 limitation, to measure biological information other than brain blood flow with use of a high light power or measure biological information with use of a highly sensitive photodetector such as an avalanche photodiode. In that case, the emission of a light pulse group 8b does not necessarily need to be repeated more than once within one frame period. Biological information may be detected by irradiating the target part 6 only once with a light pulse group 8b within one frame period.

The photodetector 2 according to Embodiment 1 includes the electronic shutter, which switches between storing signal charge and not storing signal charge, and the storage section 4. The electronic shutter is a circuit that controls the storage and discharge of signal charge generated by the photoelectric conversion element 3.

With continued reference to FIG. 1B, an example of operation of the photodetector 2 is described. The photoelectric conversion element 3 photoelectrically converts a component of light included in the falling period 13 of the last reflected light pulse in a reflected light pulse group 19b returning from the target part 6 due to a light pulse group 8b. After that, in reaction to control signals 16a and 16c, the photodetector 2 accumulates the signal charge 18 in the storage section 4. For example, the signal charge 18 is accumulated for a certain period of time Tₛ. Tₛ may for example be 11 ns or longer and 22 ns or shorter. After the period of time Tₛ₁ has elapsed, in reaction to the control signals 16a and 16c from the control circuit 7, the photodetector 2 selects the drain 12 and releases an electric charge from the photoelectric conversion element 3.

Therefore, within one frame period, a component of light included in the falling period 13 of the last reflected light pulse in each reflected light pulse group 19b is accumulated as one frame the signal charge 18 in the storage section 4. After the end of one frame period, the signal charge 18 is outputted as an electric signal 15 to the control circuit 7. The electric signal 15 includes the information on brain blood flow.

After the emission of a plurality of light pulse groups 8b, ambient noise may be measured by keeping the electronic shutter open and closed for the same length of time and the same number of times in the absence of light emission. The S/N ratios of the signals can be improved by subtracting the value of the ambient noise from each of the signal values.

As mentioned above, the photodetector 2 according to Embodiment 1 may be an image sensor including, for each pixel, a photoelectric conversion element 3, a storage section 4, and an electronic shutter that switches between storing signal charge and not storing signal charge in the storage section 4. In this case, the image sensor includes a plurality of photodetection cells arrayed two-dimensionally. Each of the photodetection cells accumulates, as the signal charge 18, a component of light included in the falling period of the last reflected light pulse in each reflected light pulse group 19b. Furthermore, each of the photodetection cells outputs, as the electric signal 15, an electric signal corresponding to a total amount of signal charge accumulated within a first period. This makes it possible to acquire biological information about the blood flow of the target part 6 as a moving image including a plurality of frames.

Next, a degree of modulation according to Embodiment 1 is described. A comparison is made here between a degree of modulation in a case where a single light pulse is used (see FIG. 2B) and a degree of modulation in a case where a light pulse group is used (see FIG. 3B).

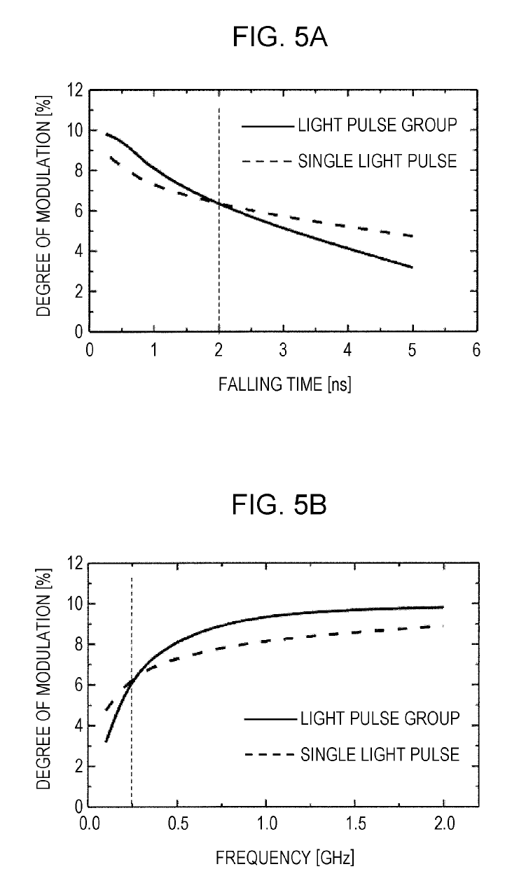

FIG. 5A is a diagram showing examples of a relationship (dashed line) between a degree of modulation in a case where a single light pulse is used and the falling time τ of the light pulse and a relationship (solid line) between a degree of modulation in a case where a light pulse group is used and the falling time τ of the last light pulse of the light pulse group. In the example shown in FIG. 5A, when τ=2 ns or shorter, the degree of modulation in a case where a light pulse group is used is higher than the degree of modulation in a case where a single light pulse is used. For example, when τ=0.25 ns, a degree of modulation of approximately 10% is attained by using a light pulse group. When τ=2 ns, the two degrees of modulation takes on substantially the same value. It is found that when τ=2 ns or longer, the degree of modulation in a case where a single light pulse is used is higher than the degree of modulation in a case where a light pulse group is used.

FIG. 5B is a diagram showing examples of a relationship (dashed line) between the degree of modulation in a case where a single light pulse is used and the repetition frequency f of light pulses and a relationship (solid line) between the degree of modulation in a case where a light pulse group is used and the repetition frequency f of light pulses. FIG. 5B is a diagram obtained by converting the falling time t represented by the horizontal axis of FIG. 5A into the repetition frequency of light pulses f=0.5/τ. It turned out that when the frequency f is 0.25 GHz or higher, the degree of modulation in a case where a light pulse group is used is higher than the degree of modulation in a case where a single light pulse is used.

The inventors found that, as shown in FIGS. 5A and 5B, a higher degree of modulation is more advantageously attained by using a light pulse group having a short falling period τ, i.e. a high frequency f. Use of such a light pulse group makes it possible to increase the proportion of a component of light including information on brain blood flow included in light from the target part 6.

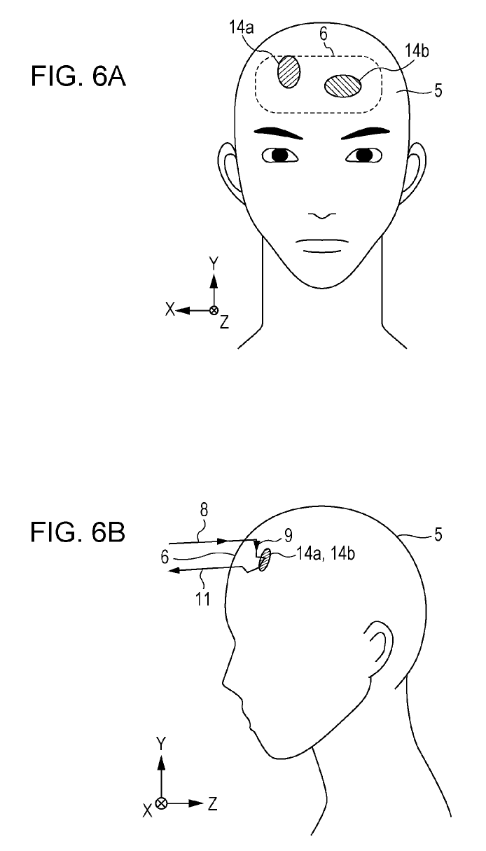

Next, the superimposition of the information on brain blood flow onto the electric signal 15 is described with reference to FIGS. 6A and 6B.

FIG. 6A is a front view showing changes in blood flow that are present in the interior of a target part 6. FIG. 6B is a cross-sectional view, taken along a Y-Z plane, showing the changes in blood flow that are present in the interior of the target part 6. FIGS. 6A and 6B show regions 14a and 14b of brain blood flow located in the interior at a depth of approximately 10 to 18 mm from the surface of the target part 6, which is a forehead. Attention is paid to the optical path through which the light 8 enters the target part 6 and is detected as the internally-scattered light 9 by the photodetector 2. The internally-scattered 9, albeit depending on a blood flow distribution, passes through the regions 14a and 14b. Furthermore, the internally-scattered light 9 is repeatedly scattered or absorbed to come out of the target part 6. This superimposes the information on brain blood flow onto the falling period 13 of the last reflected light pulse in a reflected light pulse group 19b returning from the target part due to each light pulse group 8b.

Next, a method for acquiring a distribution that indicates changes in blood flow in the target part 6 is described.

First, the control circuit 7 causes the photodetector 2, which is an image sensor, to output the following first and second image signals. The first image signal represents a two-dimensional distribution of a total amount of the signal charge 18 accumulated in the plurality of photodetection cells during the first period. The second image signal represents a two-dimensional distribution of a total amount of the signal charge accumulated in the plurality of photodetection cells during a second period preceding the first period.

Next, the signal processing circuit 30 receives the first and second image signals from the photodetector 2. After that, the signal processing circuit 30 generates a difference image corresponding to a difference between an image represented by the first image signal and an image represented by the second image signal.

The difference image is equivalent to a distribution that indicates changes in brain blood flow in the target part 6. It is assumed herein that the difference image is an image that uses the second image signal as a reference value and displays an increase or decrease in the first image signal from the reference value. When the signal processing circuit 30 receives the second image signal only once and repeatedly receives the first image signal every one-frame cycle, a moving image of a distribution that indicates changes in blood flow in the target part 6 is obtained.

Next, a method for improving the S/N ratio of the information on brain blood flow is described.

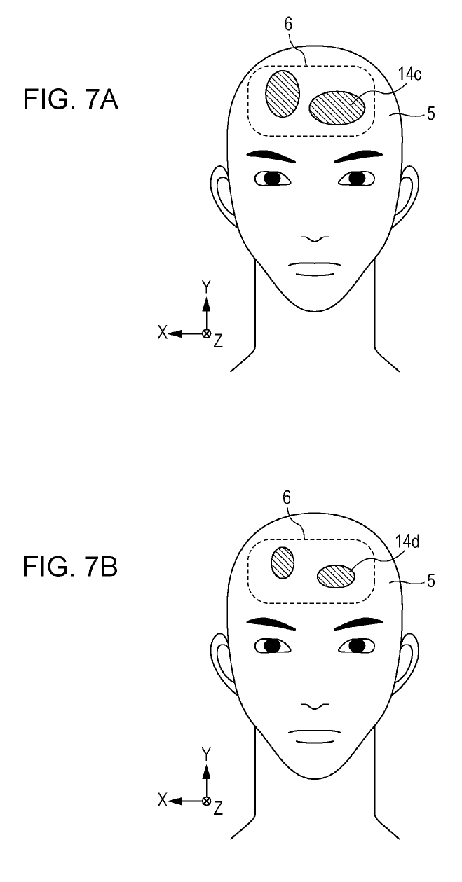

FIG. 7A is a diagram schematically showing changes in blood flow in the interior of the target part 6 as obtained from an electric signal detected in the falling period 13 of the last reflected light pulse in each reflected light pulse group 19b. FIG. 7B is a diagram schematically showing changes in blood flow in the interior of the target part 6 as image-corrected by image computations.

The signal processing circuit 30 generates blood flow information on the interior of the target part 6 with reference to the electric signal 15 corresponding to the amount of the signal charge 18. The signal charge 18 includes the blood flow information on the interior of the target part 6.

The two-dimensional image in FIG. 7A represents the distribution of the region 14c of change in brain blood flow. The region 14c of change in brain blood flow is in a spread state due to scattering of brain blood flow in the interior. To address this problem, the signal processing circuit 30 makes an image correction by guessing the scattering state through a diffusion equation or a Monte Carlo analysis. By so doing, the signal processing circuit 30 generates a two-dimensional image corresponding to a distribution of a region 14d of change in brain blood flow such as that shown in FIG. 7B. This two-dimensional image is a desired image that indicates changes in brain blood flow.

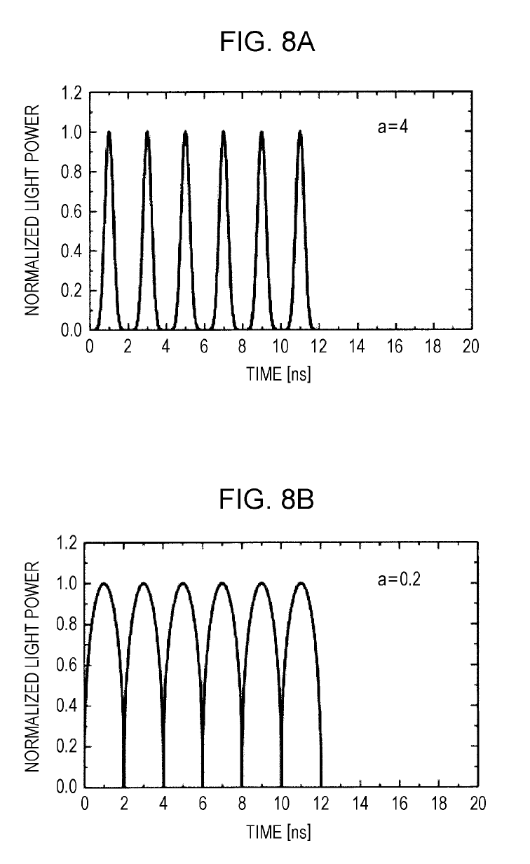

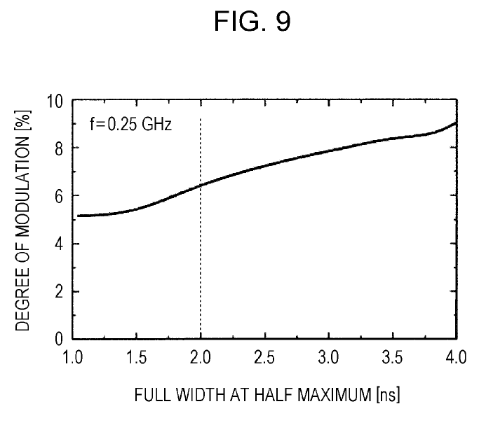

A relationship between the full width at half maximum and degree of modulation of each light pulse in a case where a light pulse group is used is described with reference to FIGS. 8A and 8B and FIG. 9. The following description assumes that the control circuit 7 causes the light source 1 to emit a light pulse group whose normalized power is expressed as P^{(a)}(f, t)=[0.5{1−cos(2πft)}]^{a}. Note here that a is a real number of greater than 0. The full width at half maximum of each light pulse can be changed by changing the parameter a.

FIG. 8A is a diagram showing an example of a time distribution of a light pulse group of P^{(a)}(f, t) where f=0.5 GHz and a=4. FIG. 8B is a diagram showing an example of a time distribution of a light pulse group of P^{(a)}(f, t) where f=0.5 GHz and a=0.2. In a case where a=1, a light pulse group such as that shown in FIG. 3A is emitted. In the example shown in FIG. 8A, the full width at half maximum of each light pulse is 0.52 ns. In the example shown in FIG. 8B, the full width at half maximum of each light pulse is 1.78 ns. As shown in the examples of FIGS. 8A and 8B, the larger a becomes, the shorter the full width at half maximum of each light pulse becomes; the smaller a becomes, the greater the full width at half maximum of each light pulse becomes. When a=1, the full width at half maximum of each light pulse is (0.5/f) seconds. When a>1, the full width at half maximum of each light pulse is smaller than (0.5/f) seconds, and when a<1, the full width at half maximum of each light pulse is greater than (0.5/f) seconds. The full width at half maximum of each light pulse is shorter than the cycle period (1/f) of light pulses regardless of the value of a.

FIG. 9 is a diagram showing an example of a relationship between the full width at half maximum and degree of modulation of each light pulse of a light pulse group of P^{(a)}(f, t) where f=0.25 GHz. As mentioned above, the full width at half maximum of a light pulse can change according to the parameter a. In a case where a=1, the full width at half maximum of each light pulse is (0.5/f)=2.0 ns. As shown in FIG. 9, the degree of modulation increases as the full width at half maximum of each light pulse becomes greater. That is, in a case where the repetition frequency of light pulses is fixed, a high degree of modulation is more advantageously attained when the full width at half maximum of each light pulse is great. That is, increasing the full width at half maximum of each light pulse makes it possible to increase a component of light including information on brain blood flow included in light from the target part 6. If 0.5/f≤pw<1/f is satisfied where pw is the full width at half maximum of each light pulse, a higher degree of modulation can be attained than in a case where a=1. In Embodiment 1, this condition is satisfied if a>1.

### EMBODIMENT 2

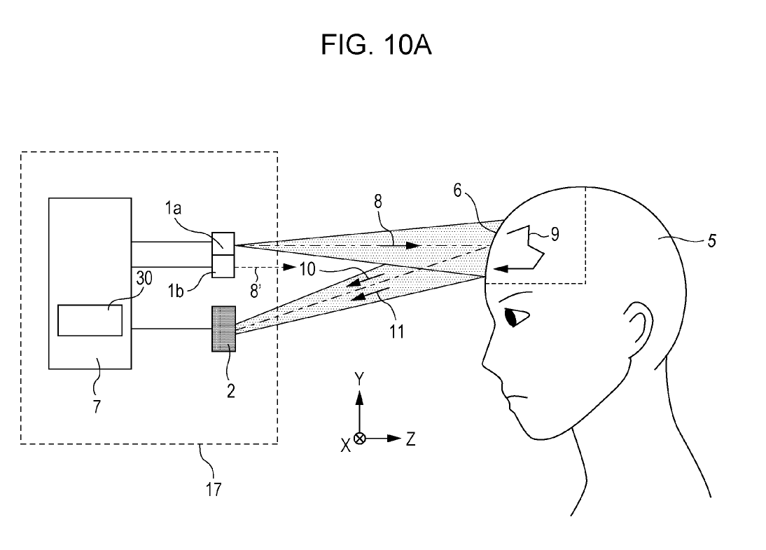

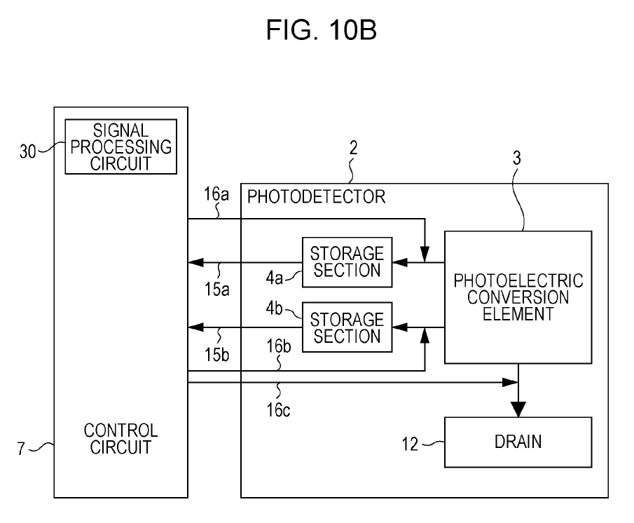

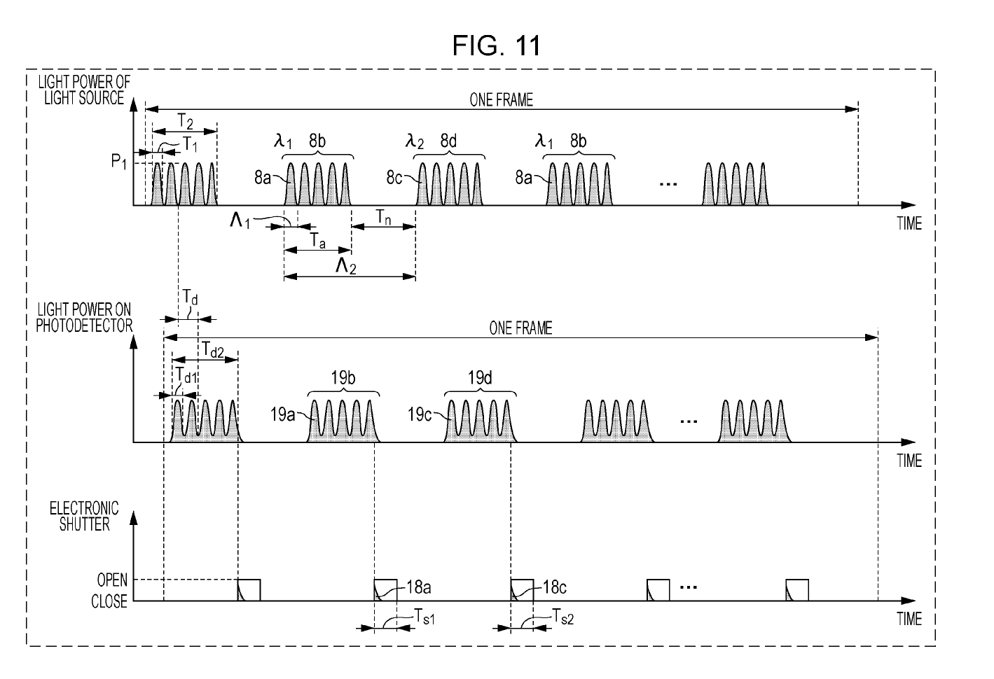

Next, a biological measuring device according to Embodiment 2 of the present disclosure is described with reference to FIGS. 10A and 10B and FIG. 11 with a focus on differences from the biological measuring device 17 according to Embodiment 1.

FIG. 10A is a schematic view for explaining a configuration of a biological measuring device 17 according to Embodiment 2 and the way in which a biological measurement is carried out. FIG. 10B is a diagram schematically showing an internal configuration of a photodetector 2 according to Embodiment 2 and the flow of electric signals and control signals.

FIG. 11 is a diagram schematically showing a time distribution (upper row) of light pulse groups 8b and 8d, a time distribution (middle row) of a light power that is detected by the photodetector 2, and the timing and charge storage (lower row) of an electronic shutter according to Embodiment 2.

The biological measuring device 17 according to Embodiment 2 differs from the biological measuring device 17 according to Embodiment 1 in that the light source 1 is a multiwavelength light source that emits light pulse groups 8b and 8d of different wavelengths in sequence.

The light source 1 is composed of a plurality of light-emitting elements 1a and 1b arranged side by side in a Y direction. The light-emitting element 1a emits light of a first wavelength range, and the light-emitting element 1b emits light of a second wavelength range that is different from the first wavelength range. The light-emitting elements 1a and 1b are for example laser chips.

The absorptance of oxyhemoglobin and deoxyhemoglobin varies, for example, at wavelengths of λ₁=750 nm and λ₂=850 nm. Therefore, computing two electric signals respectively obtained by using these two wavelengths makes it possible to measure the proportions of oxyhemoglobin and deoxyhemoglobin in the target part 6.

When the target part 6 is a forehead area of the head of a living body, the amount of change in brain blood flow in the frontal lobe, the amounts of change in oxyhemoglobin concentration and deoxyhemoglobin concentration, or the like can be measured. This makes sensing of information such as emotions possible. For example, in a centered state, there occur an increase in brain blood flow volume, an increase in amount of oxyhemoglobin, and the like.

Various combinations of wavelengths are possible. At a wavelength of 805 nm, the rates of absorption of oxyhemoglobin and deoxyhemoglobin become equal. Therefore, in view of the biological window, for example, a combination of a wavelength of not shorter than 650 nm and shorter than 805 nm and a wavelength of longer than 805 nm and not longer than 950 nm may be used. In this case, the control circuit 7 causes the light source 1 to emit one or more light pulse groups 8b and 8d by alternately repeatedly emitting a light pulse group 8b of a wavelength of not shorter than 650 nm to shorter than 805 nm and a light pulse group 8d of a wavelength of longer than 805 nm to not longer than 950 nm.

A wavelength of 805 nm may be used in addition to the two wavelengths. In a case where three wavelengths of light are used, three laser chips are needed; however, since information on the third wavelength is obtained, utilizing the information may make computations easy.

The photodetector 2 of the biological measuring device 17 according to Embodiment 3 includes an electronic shutter that switches between storing signal charge and not storing signal charge and two storage sections 4a and 4b. The light-emitting element 1a emits a light pulse group 8b of a wavelength λ₁. The photoelectric conversion element 3 photoelectrically converts a component of light included in the falling period 13 of the last reflected light pulse in a reflected light pulse group 19b returning from the target part 6 due to the light pulse group 8b of the wavelength λ₁. After that, in reaction to control signals 16a, 16b, and 16c, the photodetector 2 selects the storage section 4a and accumulates a first signal charge 18a for a period of time Tₛ₁ of, for example, 11 to 22 ns. After the period of time Tₛ₁ has elapsed, in reaction to the control signals 16a, 16b, and 16c from the control circuit 7, the photodetector 2 selects the drain 12 and releases an electric charge from the photoelectric conversion element 3.

After this, the biological measuring device 17, replacing the light-emitting element 1a with the light-emitting element 1b, similarly emits a light pulse group 8b of a wavelength λ₂. The photoelectric conversion element 3 photoelectrically converts a component of light included in the falling period 13 of the last reflected light pulse in a reflected light pulse group 19d returning from the target part 6 due to the light pulse group 8d of the wavelength λ₂. After that, in accordance with the control signals 16a, 16b, and 16c, the photodetector 2 selects the other storage section 4b and accumulates a second signal charge 18c for a period of time Tₛ₂ of, for example, 11 to 22 ns. The period of time Tₛ₂ may for example be 11 ns or longer and 22 ns or shorter. After the period of time Tₛ₂ has elapsed, in reaction to the control signals 16a, 16b, and 16c from the control circuit 7, the photodetector 2 selects the drain 12 and releases an electric charge from the photoelectric conversion element 3.

Thus, a component of light included in the falling period 13 of the last reflected light pulse in a reflected light pulse group 19b returning from the target part 6 due to each light pulse group 8b of the wavelength λ₁ is accumulated as the first signal charge 18a in the storage section 4a during one frame period. After the end of this frame period, the first signal charge 18a is outputted as a first electric signal 15a to the control circuit 7. The first electric signal 15a includes the information on brain blood flow of the wavelength λ₁.

Further, a component of light included in the falling period 13 of the last reflected light pulse in a reflected light pulse group 19d returning from the target part 6 due to each light pulse group 8d of the wavelength λ₂ is accumulated as the second signal charge 18c in the storage section 4b during the same frame period. After the end of this frame period, the second signal charge 18b is outputted as a second electric signal 15b to the control circuit 7. The second electric signal 15b includes the information on brain blood flow of the wavelength λ₂.

On the basis of the first and second electric signals, the signal processing circuit 30 generates two pieces of image information, respectively. Then, from these two pieces of image information, the signal processing circuit 30 generates, for example, an image of two two-dimensional concentration distributions of oxyhemoglobin and deoxyhemoglobin as an image that indicates changes in brain blood flow.

Next, a biological measuring device according to a modification of Embodiment 2 is described.

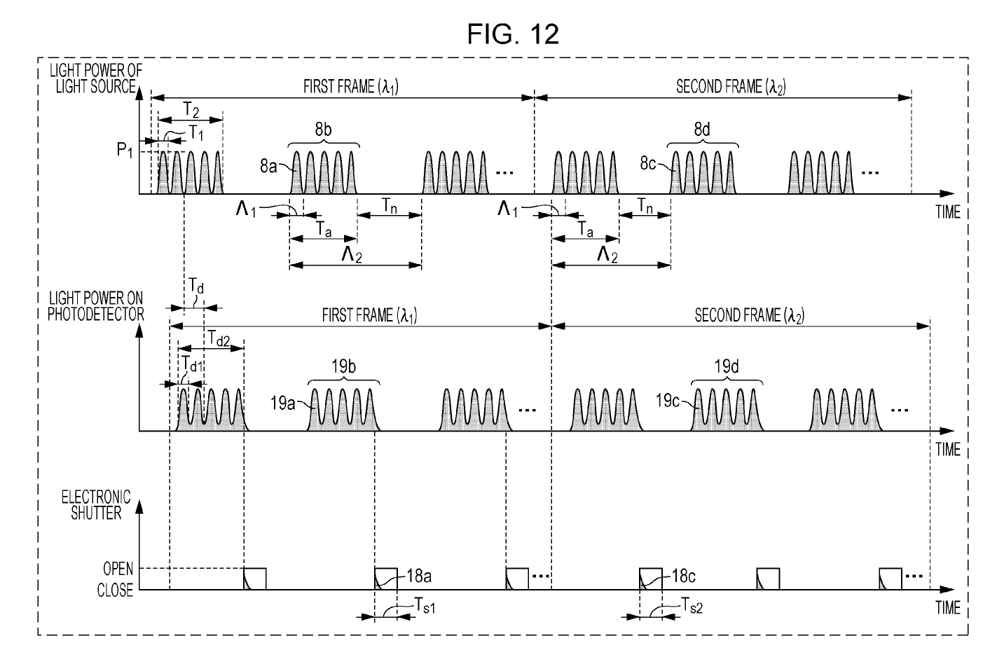

FIG. 12 is a diagram schematically showing a time distribution (upper row) of light pulse groups 8b and 8d, a time distribution (middle row) of a light power that is detected by the photodetector 2, and the timing and charge storage (lower row) of the electronic shutter according to a modification of Embodiment 2 of the present disclosure.

The control circuit 7 causes the light source 1 to emit one or more light pulse groups by emitting a light pulse group 8b of either one of a wavelength of not shorter than 650 nm to shorter than 805 nm and a wavelength of longer than 805 nm to not longer than 950 nm at least once within a first period and emitting a light pulse group 8d of the other one of the wavelengths at least once within a second period that follows the first period.

In this example, the acquisition of blood flow information by light of the wavelength λ₁ and the acquisition of blood flow information by light of the wavelength λ₂ are performed in different frame periods. In this example, either one of the two storage sections is used.

During a first frame period, a light pulse group 8b having a first wavelength λ₁ is repeatedly emitted. An electric charge of a component of light that is equivalent to the rear-end part of each light pulse group 8b is accumulated in a first one of the two storage sections. After the end of the first frame period, the control circuit 7 reads out a first electric signal from the first storage section.

During a second frame period, a light pulse group 8d having a second wavelength λ₂ is repeatedly emitted. An electric charge of a component of light that is equivalent to the rear-end part of each light pulse group 8d is accumulated in the first storage section. After the end of the second frame period, the control circuit 7 reads out a second electric signal from the first storage section.

Next, results of an experiment are explained. The experiment was conducted for the purpose of evaluating the quality of an experimental signal including the falling time of a light pulse group emitted from the light source 1. In the experiment, the light source 1 emitted a light pulse group by being supplied with a driving current on which a high-frequency component had been superimposed. In the experiment, the target part was not irradiated. In the experiment, the photodetector 2 directly detected the light pulse group. In this example, the light source 1 used was an LD of a wavelength of 785 nm, and the photodetector 2 used was a PIN photodiode.

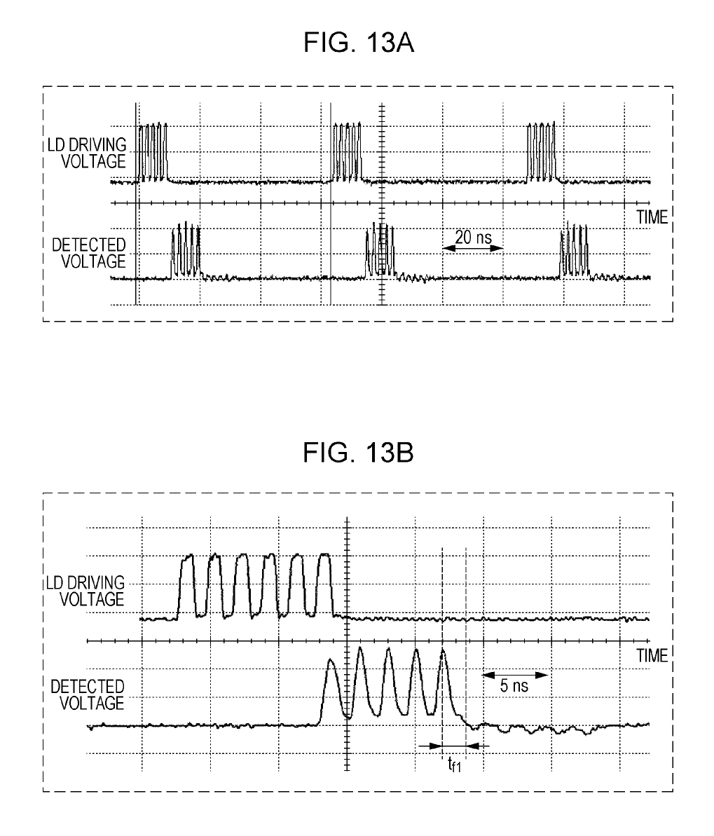

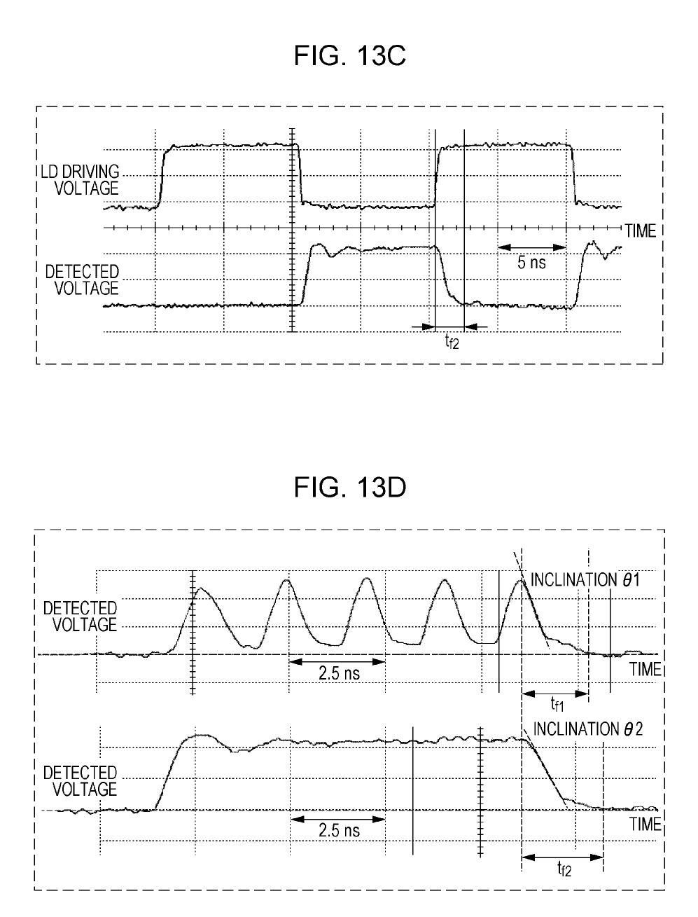

FIG. 13A is a diagram showing examples of a time distribution (upper row) of LD driving voltage and a time distribution (lower row) of detected voltage of a photodetector at the time of emission of a plurality of light pulse groups. The waveform of the LD driving voltage corresponds to the waveform of a light pulse group emitted. In the example shown in FIG. 13A, pulse groups are emitted at intervals of 64 ns. The full width at half maximum of each pulse in each pulse group is 1 ns. Pulses are emitted at intervals of 2 ns. The frequency of each pulse is 0.5 GHz. The falling time of each pulse is 1 ns.

FIG. 13B is a diagram showing an example of a time distribution of one of the plurality of light pulse groups shown in FIG. 13A. The falling time of the last pulse of a pulse group of photodetection voltage in the lower row of FIG. 13B was measured as t_{f1}=1.7 ns.

FIG. 13C is a diagram showing examples of a time distribution (upper row) of LD driving voltage and a time distribution (lower row) of detected voltage of a photodetector in a case where a single light pulse is emitted. The full width at half maximum and falling time of a single light pulse in the upper row of FIG. 13C are the same as the full width at half maximum and falling time of a light pulse group in the upper row of FIG. 13B. The falling time of the last pulse of a pulse group of photodetection voltage in the lower row of FIG. 13C was measured as t_{f2}=2.2 ns.

FIG. 13D is an enlarged view of temporal changes in detected voltage in the lower row of FIG. 13B and temporal changes in detected voltage in the lower row of FIG. 13C. It was found that in a case where a light pulse group is used, the inclination θ1 of the falling edge of the last pulse is steeper than the inclination θ2 of the falling edge of a pulse in a case where a single light pulse is used. As a result, the falling time t_{f1} was shorter than the falling time t_{f2}.

Currently, a reason for this cannot be clarified in detail. Presumably, the falling time may become shortened since, due to the state of charge inside the LD, higher-speed modulation leads to a higher-speed change in amount of injected current inside the LD and leads to an increase in annihilation rate of emitted light accordingly.

As noted previously, FIG. 5A shows a comparison between degrees of modulation in a case where a light pulse group and a single light pulse have the same falling time with the same pulse width. On the other hand, the results of the experiment show that, as shown in FIGS. 13B to 13D, the falling time becomes shorter in a case where a light pulse group is used than in a case where a single light pulse is used, even when a single light pulse and a light pulse group that are emitted have the same pulse width.

Furthermore, according to FIG. 5A, the degree of modulation tends to become higher as the falling time of emitted light becomes shorter. Considering these results, the degree of modulation shown in FIG. 5A is presumed to, in actuality, give a greater advantage to a light pulse group. That is, when the falling time τ of a light pulse is short (e.g. 2 ns or shorter), the degree of modulation is higher in a case where a light pulse group is used than in a case where a single light pulse is used, so that it becomes possible to more accurately acquire information on blood flow in the interior of the target part.

In the foregoing, the biological measuring devices according to Embodiments 1 to 3 have been described. However, the present disclosure is not limited to these embodiments. A biological measuring device based on a combination of the configurations of the biological measuring devices according to the respective embodiments is also encompassed in the present disclosure and can bring about similar effects.

What is claimed is:

1. A measuring device comprising:
a light source that emits light pulse groups toward a target part of an object, the light pulse groups each including light pulses emitted sequentially;
a photodetector that detects at least a part of reflected light pulse groups, the reflected light pulse groups each including reflected light pulses sequentially returning from the target part; and
a control circuit that controls the light source and the photodetector, wherein:
the light pulse groups include a first light pulse group and a second light pulse group that is emitted by the light source after the first light pulse group,
the reflected light pulse groups include a first reflected light pulse group and a second reflected light pulse group that returns from the target part after the first reflected light pulse group,
the control circuit causes the light source to emit the light pulse groups, and
the control circuit causes the photodetector to extract a component of light included in a last reflected light pulse of the reflected light pulses included in each of the reflected light pulse groups during a falling period, the falling period being a period from a point in time at which the last reflected light pulse starts decreasing in light power to a point in time at which the last reflected light pulse finishes decreasing in light power, and to output an electric signal corresponding to the component of light.

2. The measuring device according to claim 1, wherein:
the control circuit first causes the light source to emit the first light pulse group and then, after a period of time has elapsed, causes the light source to emit the second light pulse group, the period of time being longer than a period of time from beginning to end of emission of two consecutive light pulses included in the light pulse groups.

3. The measuring device according to claim 1, wherein
the control circuit causes the light source to emit the light pulses in each of the light pulse groups at a frequency f, and
the frequency f is equal to or higher than 0.25 GHz.

4. The measuring device according to claim 3, wherein
0.5/f≤pw<1/f is satisfied where pw is a full width at half maximum of a light power of each of the light pulses included in each of the light pulse groups.

5. The measuring device according to claim 1, wherein:
the photodetector is an image sensor including photodetection cells arrayed two-dimensionally,
each of the photodetection cells accumulates the component of light as a signal charge, and
the electric signal corresponds to the signal charge accumulated by each of the photodetection cells.

6. The measuring device according to claim 1, wherein:
the first light pulse group includes light pulses having a wavelength of not shorter than 650 nm to shorter than 805 nm,
the second light pulse group includes light pulses having a wavelength of longer than 805 nm to not longer than 950 nm, and
the control circuit causes the light source to alternately emit the first light pulse group and the second light pulse group.

7. The measuring device according to claim 1, wherein:
the first light pulse group includes light pulses having a wavelength of not shorter than 650 nm to shorter than 805 nm,
the second light pulse group includes light pulses having a wavelength of longer than 805 nm to not longer than 950 nm, and
the control circuit causes the light source to emit either one of the first and second light pulse groups at least once within a first period and causes the light source to emit the other one of the first and second light pulse groups at least once within a second period that follows the first period.

8. The measuring device according to claim 1, wherein:
the light source is a semiconductor laser, and
by supplying the light source with a driving current on which a high-frequency component has been superimposed, the control circuit causes the light source to emit the light pulse groups.

9. The measuring device according to claim 1, wherein
the light source is a self-oscillation laser.

10. The measuring device according to claim 1, wherein:
the detector includes an electronic shutter, and
the control circuit closes the electronic shutter until the falling period starts and opens the electronic shutter after the falling period starts.
